(19) United States
(12) Patent Application Publication
(10) Pub. No.: US 2025/0369867 A1
(43) Pub. Date: Dec. 4, 2025

(54) MICROSCOPY-BASED MICROFLUIDIC PARTICLE CAPTURE

(71) Applicant: Atrandi Biosciences, Vilnius (LT)

(72) Inventors: Karolis Leonavicius, Vilnius (LT); Juozas Nainys, Vilnius (LT); Arnoldas Jasiunas, Vilnius (LT); Saulius Valatka, Vilnius (LT)

(21) Appl. No.: 19/302,312

(22) Filed: Aug. 18, 2025

Related U.S. Application Data
(63) Continuation of application No. PCT/EP2025/063680, filed on May 19, 2025.
(60) Provisional application No. 63/650,026, filed on May 21, 2024.

(51) Int. Cl. G01N 15/149; G01N 15/14; G01N 15/1434; G01N 33/49

(52) U.S. Cl. CPC G01N 15/149; G01N 15/1434; G01N 33/4915; G01N 2015/1402; G01N 2015/1493; G01N 2015/1497

(57) ABSTRACT

A method of sorting particles, the method comprising: introducing a population of particles into a flow cell; identifying at least one target particle among at least some particles of the population of particles; selectively polymerizing a hydrogel over at least part of the at least one target particle so as to fix the at least one target particle at a position on the flow cell.

### CROSS-REFERENCE TO RELATED APPLICATIONS

This document is a continuation of PCT Application No PCT/EP2025/063680, filed May 19, 2025, and further claims the benefit of priority to US Prov Ser. No. 63/650,026, filed May 21, 2024, the contents of which are hereby incorporated by reference in their entirety.

### BACKGROUND

Analysis of heterogeneous biological compositions is limited by the technical challenges associated with sorting these compositions into enriched or homogeneous subpopulations.

Heterogeneous cell populations, in particular, arise frequently in molecular analyses, and present challenges for analysis. Tumor cell populations, for example, often comprise a broad range of cell types from quiescent or senescent tumor cells to tumor stem cells that drive tumor proliferation. Distinguishing these subtypes, and identifying mutations that characterize each type, are valuable tasks for analysis of these populations.

This challenge is met in some cases by analyzing the compositions in bulk by, for example, generating a nucleic acid library from the composition as a whole. Alternately, some approaches comprise specific cell identification followed by fluidic driven cell sorting. Sorting cells while maintaining individual cell information, and while nonetheless amassing sufficient material for downstream analysis, remains challenging.

### SUMMARY

Provided herein are compositions and methods for target imaging, sorting and bulking without loss of individual cell information and without relying upon target-specific fluidics driven sorting. As a result of the approach to target separation, nonuniform target pools may be screened having a diversity much greater than that screened by approaches using microfluidics based sorting. Similarly, target pools or particle populations may be sorted using substantially simpler microfluidics systems, such as those that do not rely upon changing flow pressure or direction, or in some cases microvalve mediation, to separate free floating particles from one another

Accordingly, disclosed herein are methods of sorting particles, the methods comprising one or more of the following steps: introducing a population or target pool of particles or targets into a flow cell; capturing images of at least some particles of the population; identifying at least one imaged target particle among the images of the at least some particles; selectively polymerizing a hydrogel over at least part of the at least one imaged target particle (such as at least, at most, or about 20%, 30%, 40%, 50%, 75%, 90%, 95%, 99% or 100%, or a number spanned by or outside of this range) so as to fix, or thereby fixing the at least one imaged target particle at a position on the flow cell.

Target pool constituents are discarded or are optionally retained for either an immediate second round of iterative screening, or further processing, which may comprise, among other treatments, staining or re-staining the target pool, or subjecting the target pool to growth conditions so as to trigger proliferation of an encapsulated cell within the target pool.

Target particles or contents of target particle microcapsules are in some cases barcoded to preserve positional information relating to the location to which the target was attached to the surface of the flow cell. Target particles or target microcapsule contents are then released and collected for downstream analysis, which in some cases comprises correlating microcapsule contents to the image of the microcapsule used to direct the microcapsule or other target to the flow cell or other surface.

Also disclosed herein are methods of bulking microcapsule contents, comprising flowing a heterogeneous population of microcapsules across a surface, identifying a first microcapsule of the population harboring a first content such as a molecule, cell or cell population, and a second microcapsule of the population harboring the first content molecule; affixing the first microcapsule and the second microcapsule to the surface; passaging a third microcapsule not harboring the first content molecule across the surface without affixing it to the surface; and releasing the contents of the first microcapsule and the contents of the second microcapsule to be collected in bulk. Identifying in some cases comprises imaging the first microcapsule. The surface in some cases comprises oligos such as position indicative oligos, such that upon release of the first microcapsule contents, such as nucleic acids from a cell or a cell population as may arise from a single cell triggered to proliferate, the nucleic acids are labeled by the oligos so as to retain positions specific or position indicative oligo information. Upon collecting the released contents, one may sequence the nucleic acids in bulk, and using the oligo conveyed positional information, assign nucleic acids of common origin to a common microcapsule or a common position on the flow cell surface. If positional information is recovered, one may also associate the positional information to the image information used to select the microcapsule for attachment to the surface.

Also disclosed are methods of barcoding an unlabeled nucleic acid library, such as one generated from a cell population expanded in a microcapsule from a single cell precursor, comprising one or more of the steps of releasing an unbarcoded nucleic acid library or library constituent onto an array barcoded surface. for example from a microcapsule or population of microcapsules, wherein the array barcoded surface comprises barcoded oligos such as barcoded oligos indicative of positional information on the surface, and attaching the oligos, or some of the information of some of the oligos, or adding sequence of at least some of the oligos to the library. The barcoded library may then be bulked alone or with other libraries or nucleic acids for sequencing in aggregate and then the results sorted such that nucleic acids arising from a microcapsule at a position may be assigned to that position, or that microcapsule. In cases where the microcapsule is imaged prior to attachment to the surface or release of contents onto the surface, the image may be correlated with the nucleic acid reads having that position.

Similarly disclosed herein are flow cells such as those comprising an oligo array and at least one microcapsule or other nucleic acid partition bound by a hydrogel to the flow cell, wherein the microcapsule comprises an unlabeled nucleic acid library. Alternately or in combination, disclosed are flow cells comprising an oligo array and at least one nucleic acid library released from a microcapsule or other partition onto the oligo array, as well as flow cells comprising at least one microcapsule or other partition bound by a hydrogel to the flow cell at a position, wherein the microcapsule or other partition comprises an unlabeled nucleic acid library or constituent thereof, and at least one oligo bound to the microcapsule, and wherein the microcapsule or other partition harbors barcode information indicative of the position or of microcapsule or other partition contents.

Also disclosed herein are systems and methods for particle sorting by targeted hydrogel formation, so as to stabilize or fix particles or so as to impact carrier flow rate or direction. Such methods or systems effect particle sorting without relying upon targeted changes in flow rate or direction to separate a first free-floating particle from a second free-floating particle. Similarly, such methods or systems do not rely upon microvalves or carrier pump modulation to control selective carrier flow or particle flow through a linear or branched flow cell. Accordingly, simple flow cell designs, simple microfluidics pumps, and simple carrier flow directing may be used in combination with selective hydrogel formation to effect specific, accurate, and efficient particle sorting and carrier flow control. Some systems comprise a reference dataset such as an image dataset stored on a computer, as well as a computing functionality that enables comparison of a particle image to the reference dataset so as to ‘call’ the particle as corresponding to a particle of a class correlating to the reference dataset image.

The disclosure is further understood by the detailed description, examples, and the listing of claims below.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A presents a workflow schematic for “N steps” of iterative screening of a target pool to effect target capture on a surface, followed by a single target recovery step.

FIG. 1B presents a workflow schematic for particle injection into a flow cell followed by “N steps” of movement to a new flow cell area, image capture and analysis, and particle capture, followed by a single target recovery step.

FIG. 2 presents a snapshot of target capture from a target pool flowing across a flow cell.

FIG. 3 presents images of flow cells on which hydrogel polymers have been affixed.

FIG. 4 presents an exemplary system consistent with the disclosure.

FIG. 5A presents image capture data onto which a parameter gate is superimposed so as to specify particles for retention.

FIG. 5B presents a control system architecture consistent with the disclosure herein.

FIG. 6 presents a multi-flow cell system consistent with the disclosure herein, allowing for capture of target particles on multiple flow cell positions, such that upon saturation of a first flow cell position the flow cell stage may be moved and a second flow cell position may be used.

FIG. 7 presents a system consistent with the disclosure herein having a pipette port for reagent introduction and target recovery.

FIG. 8A presents monomer units of two exemplary polymers.

FIG. 8B presents a pattern of polymerized HAMA monomers of FIG. 8A.

FIG. 9 presents an image of selectively captured microcapsules.

FIG. 10 presents a microchip design.

FIG. 11 presents a detailed view of a portion of the microchip design.

FIG. 12 presents details of hydrogel spot formation.

FIG. 13 presents data related to hydrogel spot formation.

### DETAILED DESCRIPTION

Disclosed herein are compositions, systems and methods related to selective hydrogel formation in flow cell liquids. This hydrogel formation is used for target isolation from a target pool by fixing target particles to a surface, as well as for selective control of carrier flow paths through a flow cell. Targets flowing across a flow cell surface or settled onto a flow cell surface are identified, such as through imaging, and affixed to the flow cell surface through selective hydrogel encasing or hydrogel formation so as to attach to the flow cell. Targets may be subjected to analysis on the surface, or reacted to surface reagents such as oligos so that target nucleic acids or nucleic acids of targets are commonly barcoded for downstream analysis. Target pools may then be discarded, or collected (that is, not discarded) for analysis or collected to repassage over the flow cell for a second or other iterative round of selective isolation, for example following an incubation or growth treatment. Manipulation of target pool destinations is in some cases by hydrogel-mediated opening or closing of various flow cell channels. Targets may be released from the flow cell and pooled for downstream analysis, such as nucleic acid sequencing.

### Target Capture and Enrichment

Some compositions, systems and methods herein relate to target capture, such as targets encased in microcapsules or other partitions, such as a first target encapsulated in a first microcapsule or a first other partition. The disclosure is compatible with a broad range of targets, such as cells, nucleic acid libraries. cell lysates, or other biomolecules such as those that may be enclosed in or generated in microcapsules or flown freely without losing proximal integrity. Formation of microcapsule targets is disclosed in, for example, PCT Publication No WO2023/099610, published Jun. 8, 2023, which is hereby incorporated by reference in its entirety, and in PCT Publication No WO2020/255108. published Dec. 24, 2020, which is also hereby incorporated by reference in its entirety.

Targets or target pool constituents are flowed across a flow cell surface in a polymerizable carrier. The target pool constituents are imaged such that they may be distinguished from one another and so that they may be assayed for one or more distinguishing characteristics that may vary among members of the target pool, and that may be correlated to at least one image feature.

Use of any one or more of a broad range of characteristics is consistent with the disclosure herein, such as a cell morphological feature, imaged in a free-floating cell or in a cell encased in a microcapsule, or the presence or identity of microcapsule contents as may be assayed by contacting my reagents so as to generate a content-dependent nucleic acid amplicon, as may be detected using a double-strand dependent fluorophore such as SYBR Green, or as may be assayed using an antibody. fluorophore, or other marker of target identity or presence.

Target pool constituents are in some cases imaged, and targets are identified for retention. Alternately, target pool constituents are assayed for a marker indicative of contents of a target in a particle of the target pool. A target to be retained is subjected to conditions so as to trigger local polymerization of the carrier medium, so as to fix the target to the flow cell surface.

A broad range of polymerizable carriers or carriers otherwise able to form hydrogels such as degradable hydrogels are consistent with the disclosure herein. Carriers are preferably polymerizable through targeted or localized addition of external energy such as high energy light for photochemical reactions or high intensity light for localized heating, for example, so as to polymerize the carrier in proximity to both the target and the flow cell surface, affixing the target to the flow cell. As an alternative to polymerization, carriers may in some cases be locally solidified such as locally frozen in proximity to a target.

A feature of many such carriers is that they are polymerizable without harm or without substantial harm to the target, such that, for example, target biological activity is retained, or in the case of cells, cell viability is maintained. That is, in some cases surface-captured targets harbor viable cells or cells having biochemically active constituents at a frequency of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the frequency of viable cells in the targets prior to hydrogel formation. In some cases, cells exhibit a survival rate of at least 99%, at least 98%, at least 97%, at least 96%. at least 95%, at least 90%, at least 85%, at least 80%, at least 75%, at least 70%, at least 60%, at least 50%, at least 40%, at least 30%, at least 20%, or at least 10% when subjected to hydrogel formation. In some cases biomolecules exhibit a bioactivity survival rate of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% when subjected to hydrogel formation. In some cases nucleic acid molecules exhibit an effective concentration of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the concentration in an individual target particle in the target pool from which the hydrogel was generated. In some cases biomolecules exhibit an effective concentration of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the concentration in the target particle from which the hydrogel target was generated. In some cases biomolecules exhibit an effective activity of at least 99%, at least 98%, at least 97%, at least 96%, at least 95%, at least 90%, at least 85%, at least 80%, at least 75%, at least 70%, at least 60%, at least 50%, at least 40%, at least 30%, at least 20%, or at least 10% of the activity in the target pool from which the hydrogel was generated.

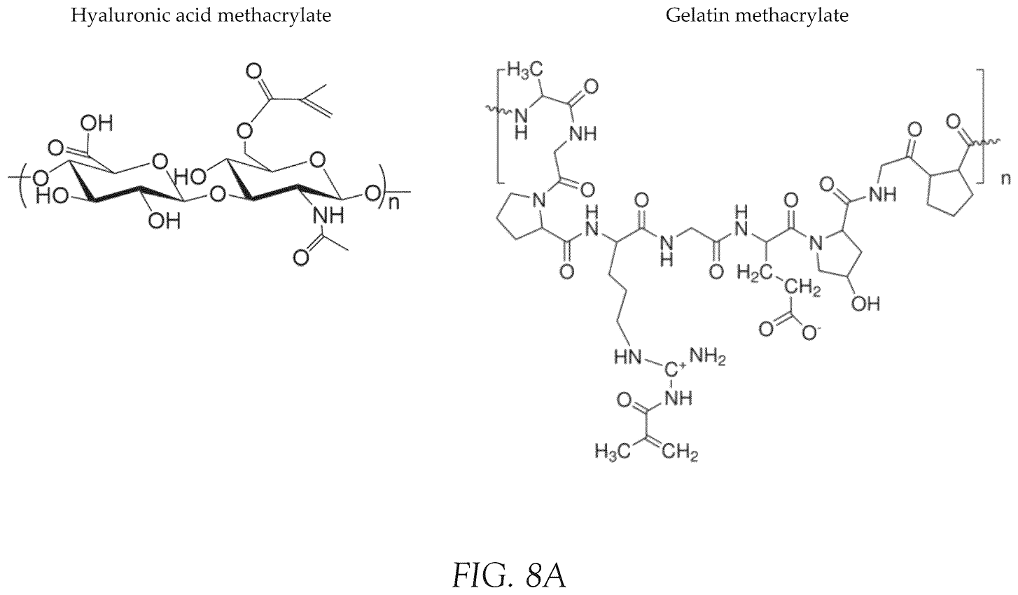

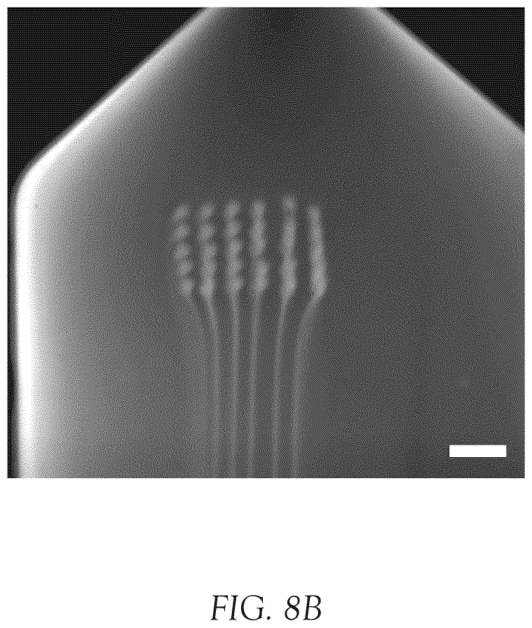

A broad range of carriers are consistent with the disclosure herein. Carriers generally comprise monomers or polymers that are locally polymerizable or may be locally solidified to form hydrogels or solid coats upon introduction of an external energy source or catalyst, such as light or electromagnetic radiation. Preferably, the resulting polymerized hydrogels or solid coats are readily degradable under physiological conditions, such as by an enzyme or by gentle heating. Exemplary carriers comprise, for example DexMAB or GelMA. Many carriers comprise carbohydrate monomers or polymers such as glucose monomers or polymers. perhaps modified by methacrylate or other acryl moiety, and capable of polymerization. Exemplary embodiments include, for example, hyaluronic acid methacrylate (HAMA) and gelatin methacrylate, each of which is readily polymerized to form a hydrogel. See, e.g., FIG. 8A and FIG. 8B.

Carriers often also comprise an energy-induced generator of oxidative stress to pass to another chemical moiety to trigger polymerization. Exemplary catalysts or polymerization triggers include, for example, an oxidative stress moiety, a free radical moiety, a phosphoryl radical, benzoyl radical (such as an LAP [Lithium phenyl (2,4,6-trimethylbenzoyl) phosphinate cleavage radical), a methyl radical as may be found on the shell polymer, a hydroxyl radical (OH·), superoxide anion (·O2—), hydrogen peroxide (H2O2), singlet oxygen (1O2), or ozone (O3), among others. In exemplary carriers, the polymerization trigger such as LAP is present at a concentration of at least, at most, about or exactly 0.01%, 0.02%. 0.05%, 0.1%, 0.2%, 0.5%, 1%, 2%, 3% or up to saturation in its aqueous carrier.

Carrier hydrogel is induced upon application of external stimulus such as chemical or electromagnetic energy. A broad range of energies or chemical treatments that allow localized hydrogel formation on a time scale sufficient to capture target particles before they depart the flow cell area are consistent with the disclosure herein. Exemplary inducing energies comprise, for example at least, at most, about or exactly 0.1 mW, 0.2 mW, 0.5mW, 1 mW, 2 mW, 3 mW, 4 mW, 5 mW, 10 mW, 20 mW or more, administered over an exposure time of at least, at most, about or exactly for example, 1 ps, 2 ps, 5 ps, 10 ps, 20 ps, 50 ps, 100 ps, 200 ps, 500 ps, 1 ns, 2 ns, 5 ns, 10 ns, 20 ns, 50 ns, 100 ns, 200 ns, 500 ns, 1 ms, 2 ms, 5 ms, 10 ms, 20 ms, 50 ms, 100 ms, 200 ms, 500 ms or greater.

Similarly, carrier solidification is in some cases induced by. for example, introduction of a source of freezing energy, such as liquid nitrogen droplets targeted to at least one particular microcapsule or other partition.

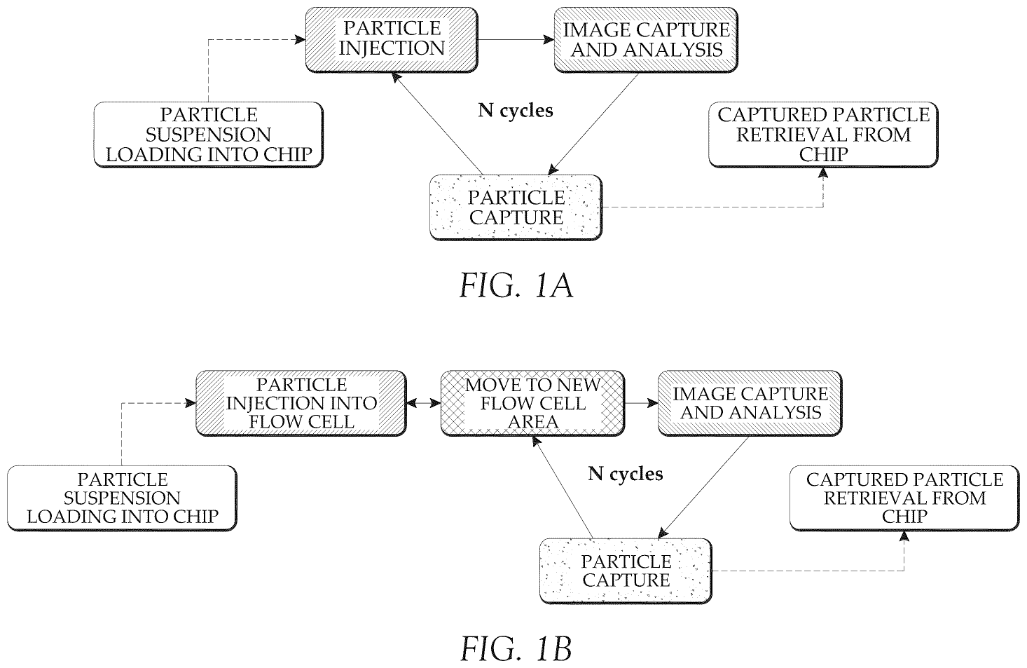

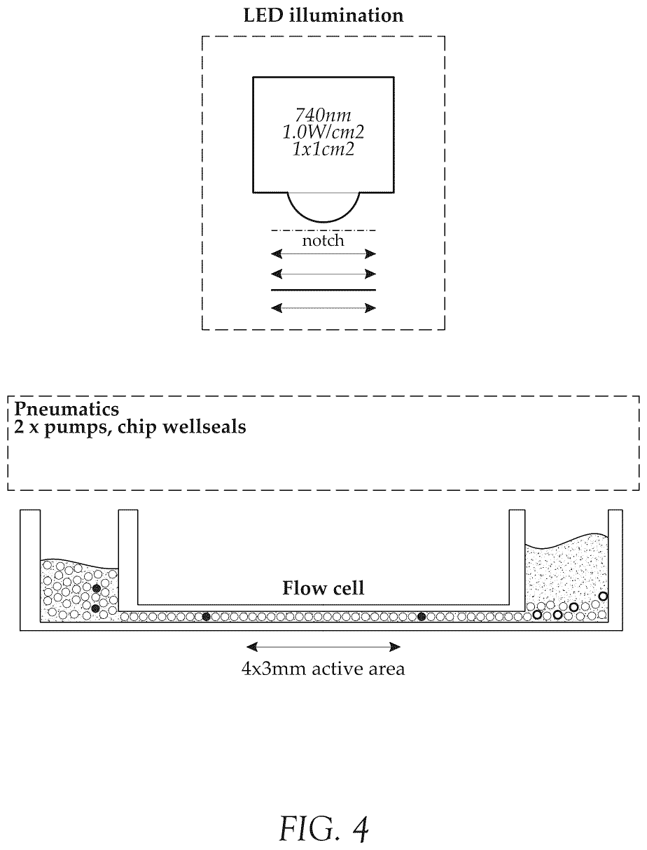

Unselected target pool constituents are in some cases subjected to flow through and discarded. Alternately, in some cases targets are recirculated through the flow cell, so as to be subjected to a second or additional round of selection, for example iterative rounds of selection. Recirculation may comprise flow through a circular path, or in some cases redirecting free particles of a target pool back through a selection chamber by changing carrier flow direction, as shown in FIG. 4, where particles may be cycled back and forth through a flow cell selection area. The second or additional round of selection is in some cases immediately subsequent to the first round. Alternately, in some cases the second or additional round of selection is performed subsequent to a treatment or treatments administered to the target pool, such as incubation under growth conditions, contacting to a fluorophore or an antibody, or nucleic acid thermocycling conditions. Alternative schematics of cyclic target screening are found in FIG. 1A and in FIG. 1B.

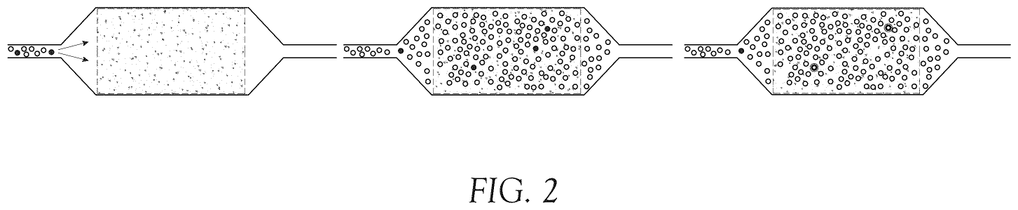

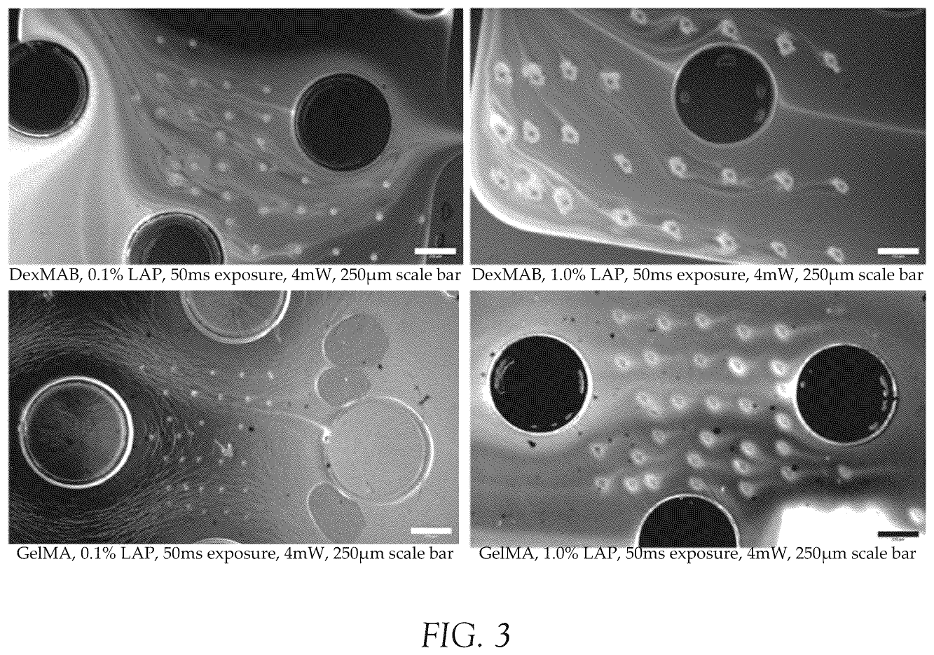

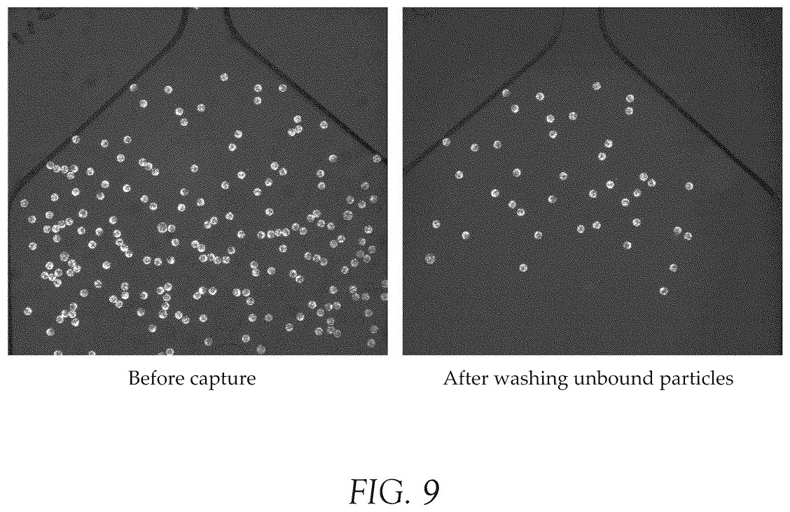

A result of one or more rounds of selection is that identified targets from a target pool are selectively attached to a surface, such that the surface is enriched for at least one target or targets, while the target pool is in some ceases depleted for targets, as shown schematically in FIG. 2 and as a specific example in FIG. 3 and FIG. 9. In some cases the surface comprises particles from the target pool of which at least 50%, 60%, 70%, 80%, 90%, 95%, 96%, 97%, 98%, 99% or even 100% are targets. Similarly, in some cases a target pool is depleted such that its target composition is reduced relative to an original target population by at least, at most, about or exactly 10%, 20%, 30%, 40%, 50%, 60%, 70%, 80%, 90%, 91%. 92%, 93%, 94%, 95%, 96%, 97%, 98% 99% or even 100%, such that targets are depleted from the target pool. Similarly, in some cases a surface has attached thereto at least, at most, about or exactly 0.0001%, 0.0002%, 0.0005%, 0.001%, 0.002%, 0.005%. 0.01%, 0.02%, 0.05%, 0.1%, 0.2%, 0.5%, 1%, 2%, 5%, 10%, 20%, 30%, 40%, 50%, 60%, 70%, 80%, 90%, 91%, 92%, 93%, 94%, 95%, 96%, 97%, 98%, 99% or even 100% of the targets of an original target pool.

Notably, target deposition or retention, and target population enrichment, is effected in some cases without any additional microfluidics such as flow-splitting microfluidics capabilities. Unlike microfluidics based sorting approaches such as FACS, identified targets do not need to be directed out of a main target pool channel into a second target channel to be separated from the target pool. Rather, separation is effected by carrier polymerization or solidification so as to affix a target to a surface such as a flow cell.

Accordingly, methods herein may be practiced on substantially streamlined microfluidics platforms relative to some platforms in the art. Methods of separating targets from target pools as disclosed herein may be practiced without redirecting targets into a channel distinct from the target pool capture or discard channel, or without applying a fluid flow to separate targets from target pools.

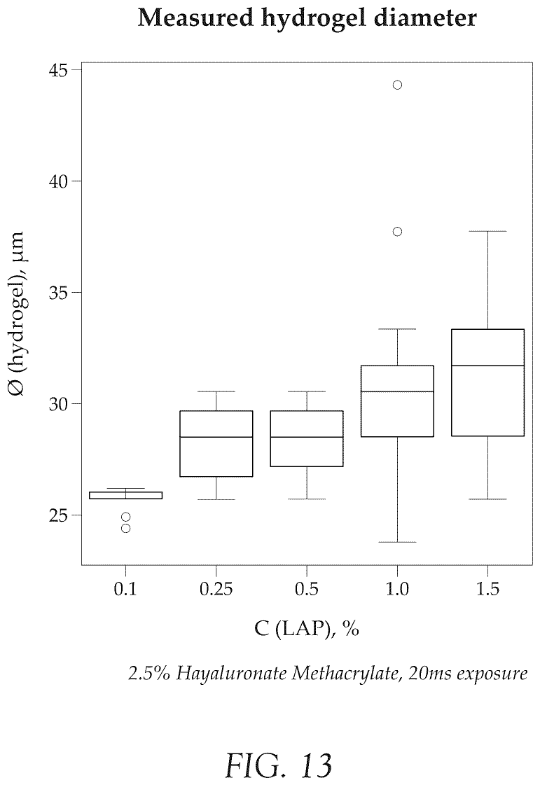

Additionally, in addition to the fluidic simplicity of the system, there is a dramatic increase in flexibility as to the types of particles in a target pool that may be assayed, as there is substantial control over hydrogel solidification size. As the methods do not reply upon channels for sorting, particles of broad structural diversity may be imaged and sorted, such as particles or targets ranging from less than 5 μm to 500 μm or greater in diameter, such as ranging from no more than, no less than, about or exactly ranging from a lower limit of 1 μm 2 μm, 5 μm, 10 μm, 20 μm, 50 μm, 100 μm, or 200 μum or more to an upper limit of 10 μm, 20 μm, 50 μm, 100 μm, 200 μm, 500 μm, 600 μm, 70 μm, 800 μm, 900 μm or 1 mm. Data relevant to hydrogel size control is seen in FIG. 13, for example. Notably, in some cases a hydrogel spot size entirely encompasses the particle which it selects or tags to a surface, while in other cases the hydrogel spot covers over at least part of the at least one imaged target particle (such as at least, at most, or about 20%, 30%, 40%, 50%, 75%, 90%, 95%, 99% pr 100%, or a number spanned by or outside of this range) so as to fix, or thereby fixing the at least one imaged target particle at a position on the flow cell.

### Target Pools/Particle Populations

A number of target pools or particle populations are consistent with the disclosure herein. Generally, target pools comprise particles that may be flowed through or suspended in a carrier such as a polymerizable carrier. Target pools are often heterogeneous, comprising both target and nontarget particles. Often, target pool comprise at least some targets that may be distinguished through observation of the target or target pool flowing through the system, such as by observing particle flow rate, mass, cell shape, cell size, cell volume, or cell surface characteristics. Alternately or in combination, targets are identified through detection of exogenously added detectors such as antibodies, nucleic acid probes or nonspecific double stranded nucleic acid assay molecules, such as those harboring a fluorophore or otherwise detectable and able to distinguish between targets and other constituents in a target pool. Targets are identified in some cases by imaging at least some of a target pool or particle population identifying a target image and correlating that target image to a particle to identify the particle as a target particle. Identifying at least one target particle in some cases comprises capturing images of at least some particles of the population, identifying a target particle image among the images of the at least some particles, and identifying a particle corresponding to the target particle image as a target particle.

The target particle image is in some cases identified by comparing to a reference set of images, for example a reference set comprising target images, and also in some cases comprising nontarget images. Targets are in some cases identified as corresponding to images that are not present in an image set, such as an image set of healthy particles or healthy cells. Target images, and their corresponding target cells or particles, may exhibit fluorescence, such as may arise from a target specific dye or fluorophore labeled binding moiety such as an antibody or antibody constituent, receptor ligand partner, organellar stain such as DAPI, a nuclear stain, a mitochondrial or vacuolar stain, or a stain indicative of metabolic activity, a stain indicative of nucleic acid amplification such as Ethidium or SYBR Green, among others. Target particle images may depict or target particles may exhibit a distinct size, shape, surface morphology, or other distinguishing feature.

Target images may be compared to a reference image set and scored as targets based upon correlation to a target image of the reference set, or based upon failure to correlate to an image of a reference set. Comparisons are in many cases automated, such as using computer based comparison approaches.

In some cases a target particle is identified by presence of a marker detected without imaging. For example, fluorescence may be assayed independently of or without reliance upon a reference image dataset. In these cases, a marker such as fluorescence, probe binding, particle size, particle flow speed, particle sedimentation properties or other property may be used to identify a target particle, either in an image of a target pool or particle population or subpopulation, or directly, without imaging, through detection of the signal such as fluorescence.

Exemplary target pools comprise nucleic acids, protein, protein or other biomolecule complexes, antibodies such as tagged antibodies, or larger moieties such as cell nuclei, organelles such as mitochondria or chloroplasts, viral particles, protein aggregates, or intact individual cells or aggregated cell clusters, such as viable intact cells. Exemplary intact cell sources include circulating cells such as cells obtained from a blood sample or other bodily fluid, disarticulated or clumped tissue cells from one or more healthy or diseased tissues, tissue harboring known or uncharacterized viral. prokaryotic or eukaryotic pathogens or contaminants. or tumor cells such as disarticulated tumor cells, alone or in combination with adjacent or distal nontumor cells. Target pool particles of broad structural diversity may be imaged and sorted, such as particles or targets ranging from less than 5 μm to 500 μm or greater in diameter, such as ranging from no more than, no less than, about or exactly ranging from a lower limit of 1 μm 2 μm, 5 μm, 10 μm, 20 μm, 50 μm, 100 μm, or 200 μm or more to an upper limit of 10 μm, 20 μm, 50 μm, 100 μm, 200 μm, 500 μm, 600 μm, 70 μm, 800 μm, 900 μm or 1 mm, or a size spanned by or outside of the listed range.

Target pools in some cases comprise any of the above particles, biomolecules or cells individually encased in a hydrogel, such as a hydrogel bead population. Similarly, target pools in some cases comprise any of the above particles, biomolecules or cells encased in a microcapsule population. In these cases, target pool constituent groupings are maintained, such that for example a cell transcriptome from a lysed cell may be enclosed in a microcapsule such as a semiporous microcapsule, resulting in the nucleic acids of the transcriptome being manipulatable as a single particle within a target pool.

Similarly, encapsulation in semiporous microcapsules facilitates processing of microcapsule contents, such as through one or more of the steps of reverse transcription, cell lysis, protease treatment, nucleic acid library formation, antibody or fluorophore binding, contacting to growth media or other processing steps. Such steps may be performed without loss of proximity of the microcapsule contents, such that they may be manipulated as a single target particle, even if they comprise a plurality of, for example, nucleic acids.

Furthermore, encapsulation in semiporous microcapsules in some cases facilitates contents release, such as enzymatic release or other release under physiological conditions. Degradation of the fixing hydrogel is also in some cases effected under physiological conditions such as enzymatically. Target microcapsule hydrogel shells and capturing hydrogels may in some cases comprise similar or identical compositions, such that hydrogel degradation to release microcapsules from the surface is concomitant with or simultaneous with microcapsule degradation, such that the microcapsule contents rather than microcapsules are released. Alternatively, in some cases microcapsules.

Accordingly, target microcapsule or other partition contents are in some cases releasable, such that they may interact with reagents on the surface such as antibodies or oligo arrays, for example conveying positional information, or such that they may be collected for bulk downstream analysis.

Microcapsules, microcapsule mediated chemical manipulation of contents, and microcapsule content release are disclosed in a number of references, such as 11,860,076, issued Jan. 2, 2024, and U.S. Pat. No. 11,958,947 issued Apr. 16, 2024, each of which is hereby incorporated by reference in its entirety.

### Target Identification

A target is identified from within a target pool through any number of approaches. For example, targets may exhibit a differential flow rate, or may sediment or settle on the flow cell surface, facilitating identification. In many cases targets are identified optically, such as through imaging of a particle to assess its appearance, or detection of a signal arising from the particle.

Optical identification variously comprises detecting single particle fluorescence, such as that associated with a fluorophore labeled antibody or probe, or with a stain or dye such as SYBR-green, ethidium bromide, or DAPI, indicative of nucleic acids such as double stranded DNA in a target particle. Similarly, a vital stain, indicative of cell viability, may be used in some cases, such as Janus green, Trypan blue or Vital red.

Identification in some cases comprises imaging of target pool constituents. Imaging variously comprises capturing cell or other target color, transparency, size, shape or surface characteristics. Images are then compared to one or more reference images, or may be assessed using machine learning, automated image calling, or artificial intelligence approaches.

Identification often occurs pursuant to target flowing across a flow cell or other surface, or subsequent to particle settling unto such a flow cell. Accordingly, imaging and subsequent target identification generally occur rapidly relative to flow rate, such that a target may be identified and its carrier locally polymerized to form a hydrogel prior to the particle departing an imaging field of view. Alternately, a particle is tracked subsequent to imaging or other detection approach, such that its carrier may be locally polymerized to form a hydrogel after detection is completed even if the target has moved substantially from the site at which the image is or was taken or other detection performed.

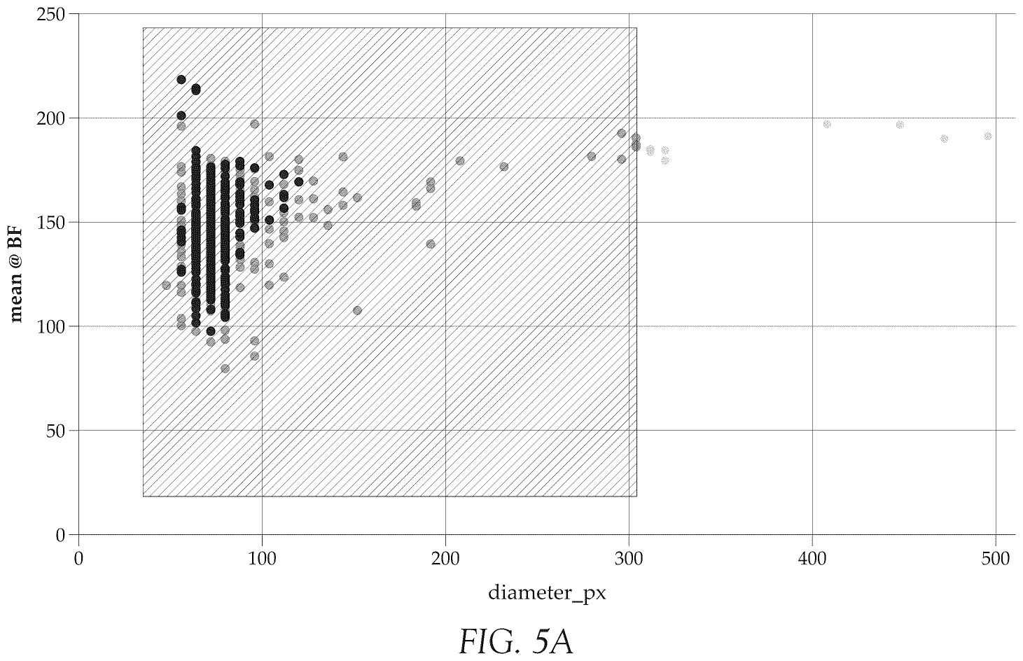

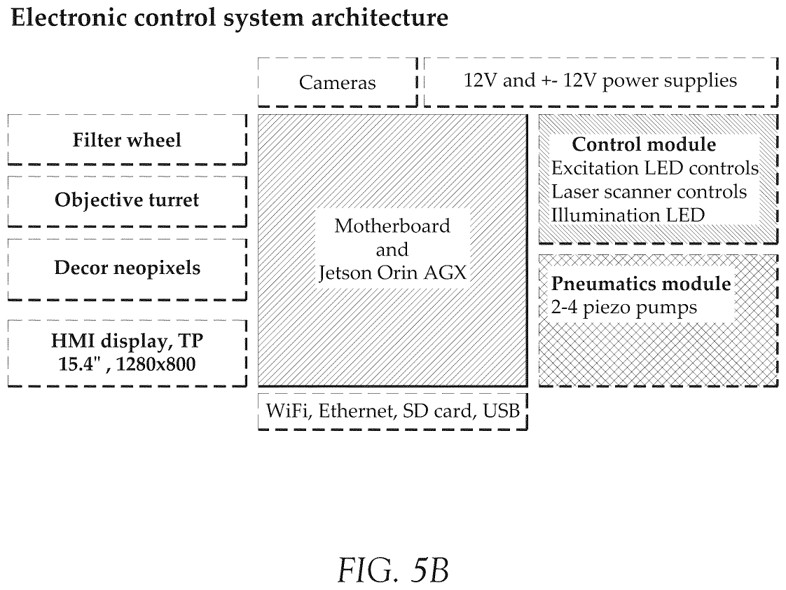

Particle identification is in some cases made pursuant to image data analysis or particle measurement. For example, once particle measurements are available. particles having particular parameters may be identified or selected for retention. Particle data may be plotted and criteria for selection, or “gates” can be set or their spanned particles identified by identifying areas of a plot corresponding to the gate or selection criteria. One may, for example, draw a shape or shapes on a plot, the area of which corresponds to the subset of the plot falling within the gate, as shown in FIG. 5A. In this example a rectangular gate specifies a subset of particles form a target pool having a diameter of no greater than 300 pixels and a mean brightness of from 10 to 250. Gates are in some cases preprogrammed such that they may be rapidly applied to datasets upon capture. Alternately, in some cases data are collected and analyzed to generate a run specific or post-imaging gate to specify particles for retention or discarding.

Subsequent to particle identification and hydrogel formation, or in some cases concurrent therewith, a location to which a target is deposited onto the flow cell or other surface may be recorded, such that the image or other detection characteristic may be correlated to data arising from analysis of the target as discussed elsewhere herein.

Notably, in various embodiments herein the location at which the target is to be deposited or fixed is not distinctly identified prior to hydrogel formation. Rather, a target is identified from a target pool, and that target is subjected to hydrogel formation. In some cases, the position of that target deposition is then recorded for subsequent analysis. Similarly, in some cases oligo sequence information indicative of location is affixed to microcapsule contents so as to convey positional information. However, this is not uniformly true across embodiments, and in many cases no record is made of the position or location at which one or more targets are deposited.

Target identification is effected using in some cases a system such as the system having components presented in FIG. 5B. As mentioned, some systems comprise a reference dataset such as an image dataset stored on a computer, as well as a computing functionality that enables comparison of a particle image to the reference dataset so as to ‘call’ the particle as corresponding to a particle of a class correlating to the reference dataset image. Systems may communicate data to a reference dataset for comparison using. for example, WiFi, ethernet, SD card or USB as indicated in FIG. 5B.

### Surfaces

Consistent with the target capture approaches and compositions herein, disclosed are surfaces suitable for target deposition. Surfaces compatible with the disclosure herein tolerate being in proximity to hydrogel formation from a carrier, and include a broad range of flow cell surfaces known in the art.

Some flow cell surfaces are unornamented. Alternately, some surfaces are decorated with markers, such as oligo markers, antibody markers, fluorophore markers or other markers. In some cases these markers convey positional information. In some cases, this information may be conveyed to a locally affixed target so as to allow that target or its constituents to be mapped back to the position where they were affixed, such as subsequent to release. Alternately or in combination, this information may be conveyed to a locally affixed target so as to allow that target's constituents to be grouped as sharing a common location identifier, such that the target constituents may be informatically mapped even if they become scattered pursuant to downstream processing.

Accordingly, in some cases a surfaces comprises an array of oligos, wherein at least some of the oligos comprise variable segments that are indicative or may be mapped back to positions on the surface. Oligos may be arrayed so as to vary continuously across positional gradients, such as cartesian X and Y axes, so that an individual oligo denotes an individual position. Alternately, oligos may be clustered into locally uniform groups, such that a particular group denotes or corresponds to a particular position or area, such as a quadrant, on the surface. In some cases, these oligo groups are separated from one another by borders lacking oligos, such that a surface presents a grid of distinct oligo regions. In some cases oligos further comprise randomer regions, such that adjacent oligos denoting a common position on a surface may nonetheless be distinguished from one another.

Oligos are in some cases tethered to the surface via linkers, such as cleavable linkers. Some such linkers are cleaved under conditions similar to or common to the conditions suitable for microcapsule and hydrogel degradation.

Nucleic acids deposited on the surface and labeled with one or more of these oligos may then carry or incorporate this positional information such that, upon sequencing, the resulting read information may be mapped to a position on the surface, or nucleic acids may be grouped or assembled such that their positional barcode information is indicative of proximity on the surface, as for example may arise from their being deposited in a common target microcapsule.

Similarly, some surfaces may comprise an array of antibodies, such as secondary antibodies barcoded to harbor positional information and targeting primary antibodies such as those used to identify or label target molecules for optical identification.

In various steps of processes herein, surfaces are bare of targets, or comprise one or a plurality of targets affixed thereto, such as by local hydrogel formation. Alternately, some surfaces comprise a first target or a first and a second target attached to the surface, for example by a hydrogel. Some surfaces comprise, for example at least, at most, about or exactly 1, 2, 5, 10, 20, 50, 100, 200, 500, 1,000, 2,000, 5,000, 10,000, 20,000, 50,000, 100,000, 200,000, 500,000, 1,000,000, 2,000,000 or more targets.

Some of said surfaces may be treated to release microcapsule contents, such as by enzymatic release or other biocompatible release, such that microcapsule contents interact with a surface directly. Such an interaction may comprise, for example, annealing to locally arrayed oligos or antibodies, such as barcoded antibodies, or binding to temporally closely or concurrently released oligos.

Thes nucleic acids are in some cases raw cell lysate products. Alternately, some such nucleic acids are processed, such as through iterative reagent delivery through the hydrogel shell of semiporous microcapsules to effect multistep sample processing, such as one or more of cell lysis, protease treatment, reverse transcription, polymerase chain reaction, or eDNA or genomic library preparation with or without barcodes or labels.

Accordingly, disclosed herein is a surface comprising, for example, an array of oligos such as oligos conveying position information, to which are annealed microcapsule released nucleic acids, for example at least, at most, about or exactly 1, 2, 5, 10, 20, 50, 100, 200, 500, 1,000, 2,000, 5,000, 10,000, 20,000, 50,000, 100,000, 200,000, 500,000, 1,000,000, 2,000,000 or more nucleic acids. Said nucleic acids are in some cases unlabeled prior to annealing with oligos of the array.

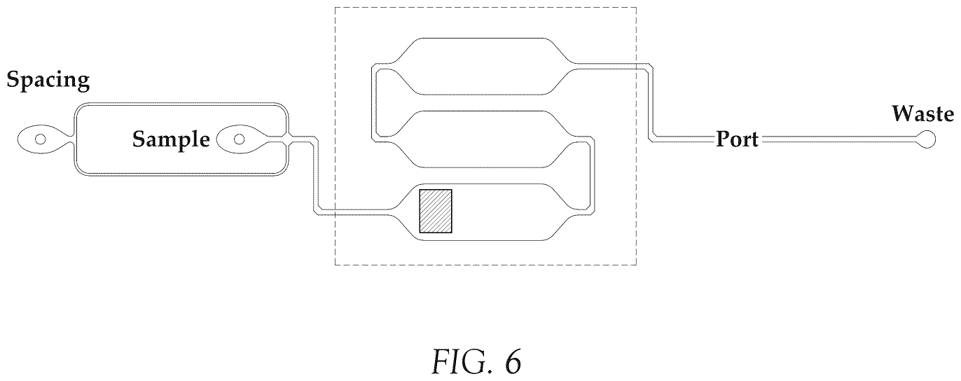

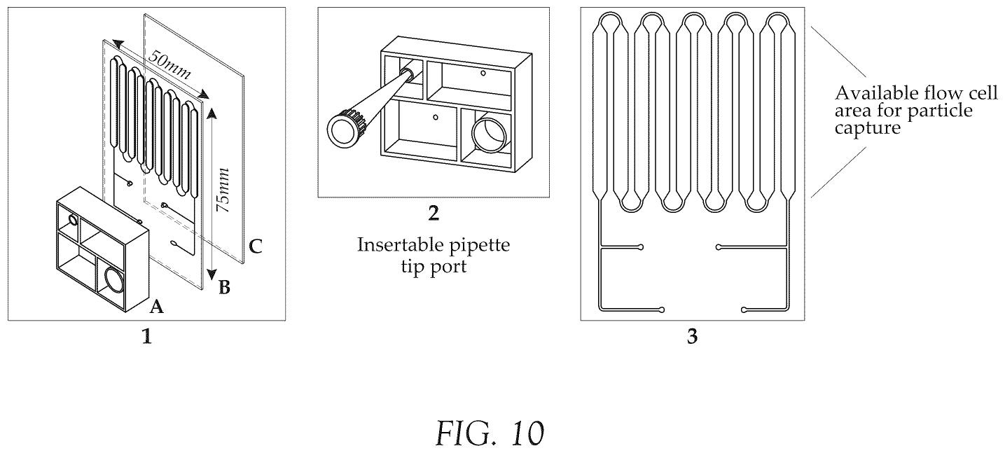

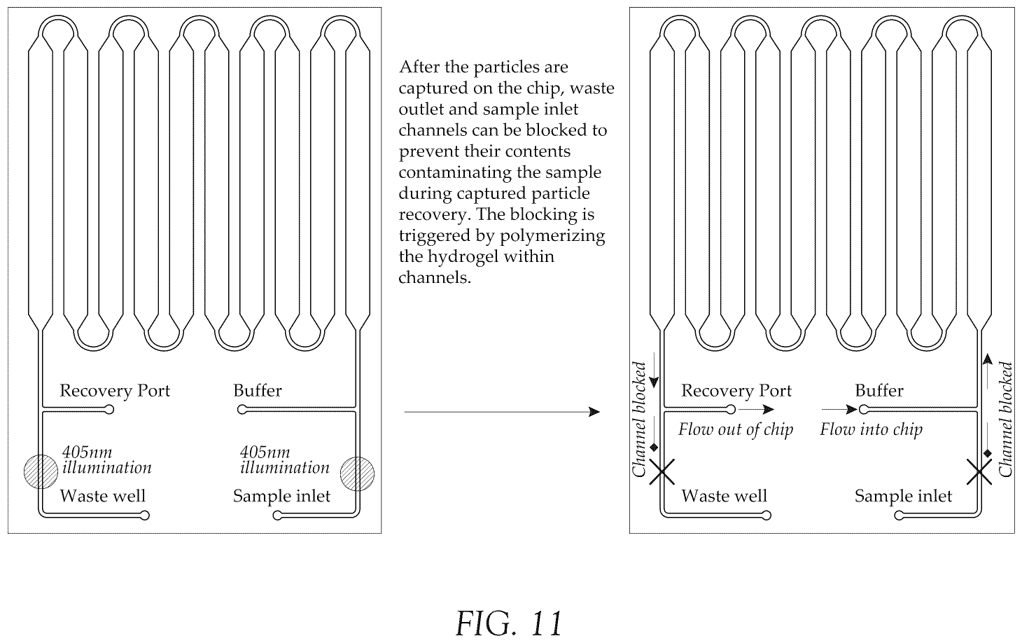

A first exemplary set of surfaces positioned within a linear flow cell is depicted in FIG. 6, while a second exemplary set of surfaces positioned withing a flow cell having branched input and output options is depicted in FIG. 10 and FIG. 11.

### Proximity Tagged Libraries

Consistent with the compositions, methods and systems elsewhere herein, disclosed are nucleic acid libraries, such as genomic or cDNA libraries, wherein library constituents are tagged to indicate a prior location of attachment to a surface, such as through a hydrogel, of a microcapsule enclosing them.

Libraries are synthesized through one or more processing steps performed in a

microcapsule, such as a semiporous microcapsule permeable to library processing reagents but impermeable to library constituents. For example, a cell is encapsulated in a microcapsule and subjected to one or more steps selected from a list comprising lysis, protease and RNase inactivation, reverse transcription, and other steps necessary or sufficient for library preparation.

Microcapsules harboring nucleic acids, or harboring cells prior to library processing, are identified among a target pool and selectively attached to a surface via light induced carrier polymerization to form a hydrogel. Processing may be performed on the surface if not done prior to attachment. In either case, processing comprises, subsequent to cell encapsulation, washing the microcapsule or microcapsule target pool in reagents in series such that the reagents are delivered to the microcapsule core, activated, and then replaced with subsequent reaction steps. These steps are performed either on free floating target pool microcapsules or on target microcapsules attached to the surface.

Subsequent to attachment, and optionally washing to remove nontarget particles from the vicinity, the microcapsules and the hydrogel attachment are degraded and contents released. As mentioned elsewhere herein, this degradation is in some cases enzymatic and performed under physiological or otherwise mild conditions. Surfaces at this stage comprise a flow cell comprising an oligo array and at least one microcapsule bound by a hydrogel to the flow cell, wherein the microcapsule comprises an unlabeled nucleic acid library.

Nucleic acids are released and react with oligos such as oligos constituting an oligo array on the surface, resulting in oligo tagged library constituents harboring positional information relating to the location of the microcapsule or the library constituents on the surface. In some cases the oligos are tethered to the surface, such that the interaction of released microcapsule contents and surface oligos results in a surface or flow cell comprising an oligo array and at least one nucleic acid library released from a microcapsule onto the oligo array. Alternatively, the oligos are released from the surface and interact with library constituents locally, such that proximity or locality tagged library constituents are generated concurrent with or subsequent to release from the surface.

Nucleic acids may then be collected from the flow cell or other surface and further processed for sequencing, by for example adding appropriate ends for short or long read sequencing techniques. The packaged libraries may then be sequenced, and sorted using oligo tag or barcode information such that reads may be grouped according to their surface location marker, allowing microcapsule contents to be reconstructed through barcoding information.

### Mapping Images to Barcode Positions in Bulked Library Sequence Data

Bulk sequence data in some cases comprises positional information correlating to the position on the surface where a particular target such as a microcapsule was deposited. Thus, this positional information may be used to assemble reads to a common target such as a common target microcapsule, and to correlate those reads to a particular position on the surface such as a flow cell surface.

Target imaging, such as that used to identify a target among a target pool and to determine that the target should be deposited onto a surface, may then be used to identify the image of the target deposited at the location correlating to the nucleic acid sequence tags.

Thus, one may obtain sequence information for a target such as a cell encapsulated in a microcapsule, and may then correlate that sequence information with an image of the target such as a cell.

Accordingly, one sees through the practice of the disclosure herein one may generate sequence data sets wherein individual reads may be correlated to cell of origin and to an image of the cell of origin. For example, a single image may correspond to at least, at most, about or exactly 1, 2, 5, 10, 20, 50, 100, 200, 500, 1,000, 2,000, 5,000, 10,000 or more than 10,000 sequence reads, or 1,000, 2,000, 5,000, 10,000, 20,000, 50,000, 100,000, 200,000, 500,000, 1,000,000, 2,000,000, 5,000,000, 10,000,000 or more bases of read information.

### Systems

Consistent with the disclosure elsewhere herein, disclosed are systems for practice of the methods and use or generation of the compositions herein.

Some such systems comprise a fluidics apparatus and a flow cell or other surface so as to flow a target pool over the flow cell or other surface. The fluidics apparatus may further comprise a reservoir for the carrier such as a polymerizable carrier from which to generate a hydrogel to fix a target to the surface. Systems may also comprise an input into which a target pool is deposited or introduced. Some systems comprise a cooling reservoir in which a cold liquid such as liquid Nitrogen may be stored and drawn upon to deliver a freezing droplet to a target particle, so as to freeze its vicinity.

A system schematic showing particle reservoirs and a detection channel is presented in FIG. 4.

Systems in some cases comprise an imaging apparatus to capture images of individual targets or sets of targets as they pass by a pre-determined field of view. Images are processed upon capture such that they may be rapidly analyzed and that targets may be identified from among the imaged subset of the target pool. Images are stored, either on the system or transmitted for remote storage, in some embodiments of the system.

Some such systems are built to integrate one or more of the components for pneumatic pressure control over plastic chip wells, a high-power multi-wavelength illumination source to excite sample fluorescence, a laser scanner for capturing selected particles and one or in some cases a pair of cameras.

Using two cameras in some systems solves the need to have high resolution brightfield images in combination with high sensitivity necessary for fluorescence and luminescence imaging. Instead of a single camera satisfying these requirements, the device may be designed to include two cameras, which could independently optimize the resolution requirements for particle detection and sensitivity requirements for their analysis.

The use of dual cameras is in some cases facilitated by efficient image registration techniques to align images during routine device use. Some techniques use laser spot imaging to establish correlation between camera images and align them.

Some systems feature the simultaneous use of a laser such as a 405 nm laser in combination with 405 nm fluorescence illumination LED. Since both light sources operate at the same wavelength, it is difficult to combine them using conventional design techniques. In this case, an approach was taken to use a notch dichroic beam splitter mirror to reflect a narrow band 405 nm light, while passing through a relatively broad spectrum of the LED. This approach has the advantage of avoiding moving parts in the setup with the disadvantage of losing some LED light, which would be reflected around the narrow 405 nm band. Other wavelengths and a broad range of intensities and exposure times are consistent with the disclosure herein.

Some devices are designed to use a laser to induce hydrogel formation, which contrasts with other technologies based on light projection techniques. Lasers allow one to reach significantly higher light intensity allowing one to reduce the time it takes to form hydrogel and increase particle capture throughput.

Some systems use simplistic laser aiming at the center of the particle to produce a hydrogel spot, which is sometimes larger than the particle to be captured. In contrast to this direct approach, some lasers can proximally target hydrogel formation near the particle, thereby reducing the chance that neighboring particles would be captured non-specifically.

Lasers or other hydrogel induction approaches of the systems herein or used in concert with the systems herein are configured to be movable or directable, such that a laser may target a specific particle or cell of a population that is identified through its image, its fluorescence or other feature discussed elsewhere herein. The laser may be positioned on a movable stage or may have a beam that can be directed or targeted, such as through a lens or mirror, to multiple positions on a flow cell. Alternately, a flow cell may be positioned on a movable stage to bring targets to a laser or other inducer path or range.

In some preferred embodiments, a focused laser spot is directed to the selected particles within a field of view using a mirror or a pair of mirrors attached to galvanometers. These mirrors control the angle at which the laser light arrives at the objective, which makes the focal position move across the field of view.

This laser scanning technique is much faster than physically moving the chip or the laser, and is preferred in some cases. Mirror directed laser delivery can point to about 300 particles per second, such as 100, 150, 200, 250, 300 or more particles per second. Stage motion, in contrast, allows one to cover several particles per second. The mirrors also allow to use most of the laser power, which allows one to have low exposure times, again making the process faster in comparison to other technologies. Alternative mirror technologies comprise micromirror arrays, which only direct a fraction of total power to a particular particle at a given time.

Thus, particles or cells identified for hydrogel affixation to a surface may be specifically targeted for hydrogel affixation using the systems herein.

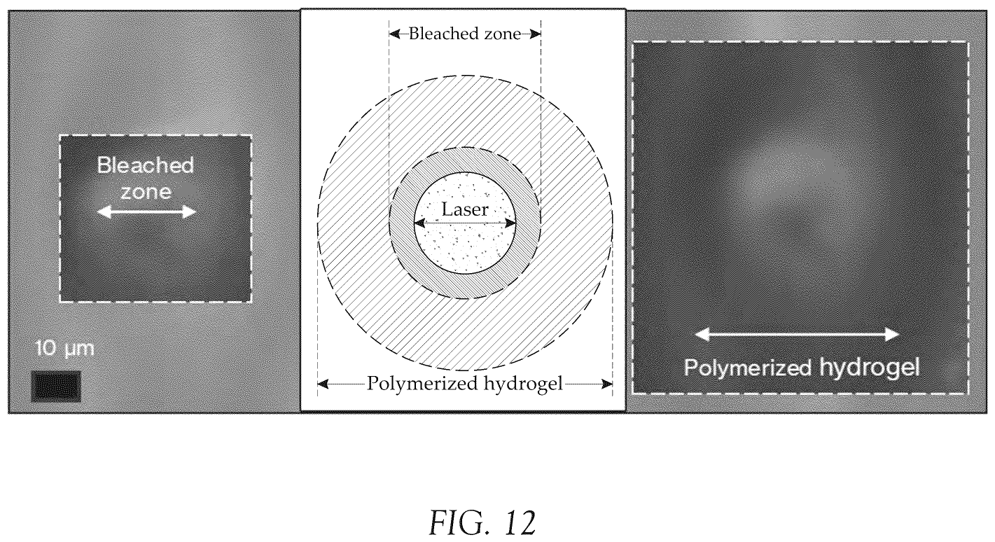

A schematic depiction of components common to many systems disclosed herein is seen at FIG. 5B, while a schematic of a laser superimposed upon a resulting hydrogel spot is shown in FIG. 12, middle panel.

Some systems rely upon use of deep learning image analysis algorithms, especially, when aiming to identify and segment irregularly shaped particles of various sizes. In contrast to conventional approaches such as Hough circles, deep learning algorithms can be trained and address more diverse biological samples than circular droplets and semi-permeable capsules (“SPCs”).

As mentioned, some systems comprise a reference dataset such as an image dataset stored on a computer, as well as a computing functionality that enables comparison of a particle image to the reference dataset so as to ‘call’ the particle as corresponding to a particle of a class correlating to the reference dataset image. Systems may communicate data to a reference dataset for comparison using, for example, WiFi, ethernet, SD card or USB as indicated in FIG. 5B.

Images such as target particle images are in some cases retained, such that data or other information correlating to a particular particle such as a cell may be correlated to the image of that particle or cell.

Upon identification of a target image, energy such as chemical or photoelectric energy. such as UV light from a laser that comprises a component of some systems, is directed to the target corresponding to the identified image, such that the carrier proximal to the target may polymerize to form a hydrogel that may tether the target to the surface.

Flow cells of the disclosed systems are in some cases designed to maximize or increase the flow cell area to fit enough captured particles for sequencing in depth.

The active flow cell area is a key chip component and is often selected or designed to be large enough to fit particle counts conventionally used for sequencing library preparation. This implies 100-10 k captured particles per experiment. Since the required area is often larger than the microscopy field-of-view, some flow cells are designed to be moved between active areas such that as one area fills up or becomes saturated by targets, a second area is made available. This is effected by moving the flow cell or changing the portion of the flow cell to be imaged, or in alternate cases is effected by moving the imaging and laser apparatus.

Moving the chip after the camera FOV fills up with captured particles may pose mechanical and algorithmic challenges. Among them, the device may include one or more of a translation stage, such as one with absolute position encoding, a translationally independent chip sealing manifold in addition to control algorithms, which may detect when a flow cell fills up and has to move to a new position.

Alternatively or in combination, some designs use a long channel with wide and narrowing sections. The wide rectangular sections facilitate particle imaging and capture to avoid the captured particles obstructing the flow of other particles in the sample. The narrow sections are designed to not be imaged and bridge the wide sections with minimal particle numbers within.

Exemplary channel configurations are seen in FIG. 6 and FIGS. 10 and 11.

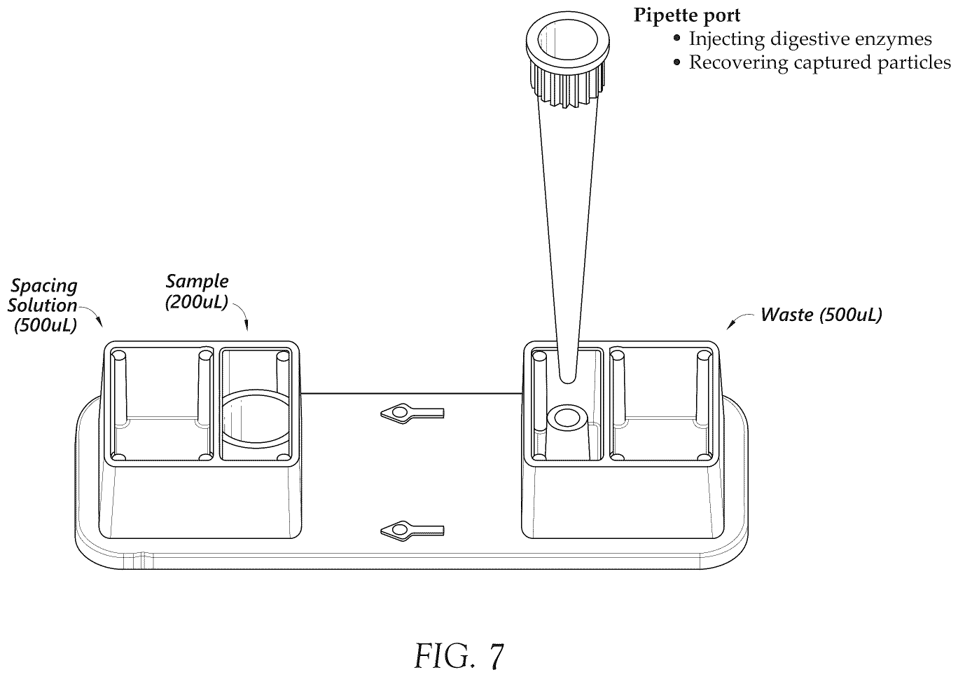

One approach to introducing samples into the chip is via chip wells. Particle suspension can be loaded into the well and then injected into the flow cell in cycles, one portion at a time. Recovering the sample after digesting the hydrogel is facilitated in some cases by using a chip designed with a pipette tip interface. which is used to extract captured particles directly from the flow cell with minimal loss. A key pipette tip interface design in some systems comprises a conical narrowing of the port, which allows to make a plastic/plastic seal with the inserted tips. Pipette interfaces are shown in FIG. 7 and in FIG. 10, panels 1 and 2.

Systems may further comprise a reservoir or an input from which a degradation component or combination of degradation components, such as enzymes that separately or in concert target microcapsule and hydrogel compositions for degradation. If libraries are processed from cells or cell populations in microcapsules subsequent to attachment to the surface, then the system may also have reservoirs or inputs to accommodate delivery of the reagents to the microcapsule on the flow cell. Similarly, delivery of reagents to trigger oligo release from the surface may also be accommodated through a reservoir or input to the system.

Systems may further comprise a flowthrough capture apparatus, such that target pool constituents not identified as targets may be captured for subsequent manipulation or repassage over the flow cell or other surface. Some target pool constituents may be re-passaged as a quality check, to give the system a further opportunity to detect targets from among the target pool. This is particularly attractive when targets are rare and difficult to obtain, such that their discarding would represent a substantial loss of resources. Alternately, this approach may be useful when targets are particularly abundant, such that targets may saturate the system's ability to affix them to the surface.

Flow through may comprise a circular channel or path, such that flow through is redirected to an input or starting point for re-imaging. Alternatively, some systems may be configured to reverse flow, such that captured target pools are flowed in a reverse direction over the surface and past the imaging apparatus again, so as to allow additional screening. Yet other systems comprise multiple instances where screening may occur, as shown in FIG. 6 and FIGS. 10-11.

Captured, or not discarded, flowthrough target pools are in some cases manipulated prior to reflow or reimaging. Target pools comprising cells and imaged to detect cell colony formation in microcapsules, for example, may be subjected to cell growth conditions prior to reimaging, such that microcapsules harboring single cells that had not proliferated may be returned to conditions suitable for cell proliferation prior to re-imaging.

A flow-through capture apparatus may further be configured to capture released microcapsule constituents or other target molecules subsequent to attachment to the flow cell or other surface. Library constituents, for example, may be collected for further packaging pursuant to sequencing. Some systems have functional component suitable for library finishing, such as library end addition. Alternately, in some systems further processing occurs off of the system.

Notably, some systems are characterized by what they do not require in order to accomplish target separation from a target pool. That is, some systems effect target segregation from target pools without flow-splitting microfluidics capabilities. Unlike microfluidics based sorting approaches such as FACS, identified targets do not need to be directed out of a main target pool channel into a second target channel to be separated from the target pool. Rather, separation is effected by carrier polymerization so as to affix a target to a surface such as a flow cell. Accordingly, system microfluidics is substantially streamlined. Furthermore, methods of separating targets from target pools as disclosed herein may be practiced without redirecting targets into a channel distinct from the target pool capture or discard channel, or without applying a fluid flow to separate targets from target pools.

As a result, in addition to the fluidic simplicity of the system, there is a dramatic increase in flexibility as to the types of particles in a target pool that may be assayed. As the system does not reply upon channels for sorting. particles of broad structural diversity may be imaged and sorted, such as particles or targets ranging from less than 5 μm to 500 μm or greater in diameter.

### Microfluidic Flow Modulation

Accordingly disclosed herein are systems and methods for regulation of fluid flow such as carrier fluid flow through microfluidic channels. Some such systems effect fluid flow modulation without using microfluidic valves to modulate at least some of the microfluidic flow. Similarly, some such systems effect fluid flow modulation without using variable pressure pump activity, that is pump activity that comprises changing a pressure from a first nonzero value to a second nonzero value. Pumps may still turn on and off, but do not need to adjust among nonzero pressure values to modulate fluid flow. Fluid flow through at least some of the channels of the system is accomplished in some cases on substantially more streamlined systems, without requiring modulating valve status or pump pressure, or in some cases in systems lacking valves or multi-pressure pumps to modulate flow through t least some channels of the system.

Some such systems and methods rely upon selective, reversible liquid solidification to block flow through a channel of the fluid system. Solidification may block a channel or channels, thereby modulating flow through the blocked channel and in some cases adjacent channels.

A fluid such as a carrier as described elsewhere herein, for example comprising a polymerizable monomer or polymer and a polymerization triggering moiety, is flowed through a channel. Exemplary polymerization triggering moieties are described elsewhere herein, and comprise oxidative stress inducing moieties such as LAP, among others, that may be induced by, for example, localized application of high energy light. Again, other approaches and compositions are disclosed herein, and some systems and methods are further consistent with compositions in the art.

Systems may further comprise a laser, a polymerization inducer injection outlet, or other moiety for localized induction of polymerization. In some cases the system comprises a glass of transparent coverslip or layer, such that the laser may be directed through the layer to the liquid in the channel. Similarly, some methods comprise projecting a laser or other inducing light through a system or channel boundary such as a lid, so as to locally induce reactive oxygen species generation and polymerization. Alternately, some systems comprise a lid having an opening, such as a closable opening, that may be used to deliver a chemical catalyst such as a reactive oxygen species or oxidative stress to a position in a channel.

Upon localized polymerization or localized hydrogel formation, the channel in which the polymerization occurs is blocked such that liquid, for example a carrier, is no longer able to flow through the channel. Consequently, in a single channel system flow is blocked, while in a multichannel or branched channel system, fluid is directed to at least one other, unblocked channel.

Consequently, a liquid can be directed to a collection reservoir rather than a waste outlet, for example, or a sample inlet can be blocked so as to protect it from contamination, among other examples. More generally, one may use the systems and methods to toggle between or among channel destination choices or block particular destinations, so as to direct fluid flow to a particular subset of destinations within a system having multiple outlets. This is effected without using microvalves or mechanical gating at the regulated channel or channels, and without modulating fluid pressure pump activity. Systems may comprise valves or a multi-pressure pump, but do not rely upon either functionality for channel flow or fluid endpoint or exit point modulation. Accordingly, some systems comprise a solid or rigid channel system lacking microfluidic valves and in which a constant fluid pressure is flown and nonetheless effectively allow selection or toggling among a plurality of channel paths or microfluidic destinations. See, for example, FIG. 11.

### Selected Particles and Selected Particle Populations

Systems and methods herein enable selection, enrichment or generation of a broad range of particles from a target population or target pool. Particles selected from a target pool may variously comprise viral particles, disaggregated or free living cells, cell clusters, SPCs or microcapsules such as those that may contain cells. Particles may fall within a common set of ‘gate’ parameters that may be broadly or narrowly defined so as to specify particular parameters or parameter ranges of selected particle populations. Notably, as particle selection is not overly reliant upon fluidic redirection for particle isolation or enrichment, selection may be performed upon a much broader range of particles than may be isolated using microfluidic carrier flow redirection for particle isolation, and subsequently enriched target particle populations may be isolated that are substantially different from those isolated using microfluidic manipulation, such as microcapsule populations, cell cluster populations, proliferating cell colony populations, or particles of at least or greater than 50 μm diameter or even 100 μm diameter, sizes at which microfluidic manipulation becomes problematic in many systems such as FACS, emulsion based or other microfluidic sorting.

Systems herein are compatible with a broad range of particles, which are in some cases significantly larger and more complex than single cells. As particle diameter may exceed 50 um (as is the case with many SPCs or microparticles), it becomes more difficult to use standard FACs systems and when it exceeds 100 μm flow sorters become practically unusable because of the nozzle dimensions or other fluidic challenges.

In addition to the mechanical constraints, larger particles are usually more complex, so an image can provide much more adequate information in comparison to a point fluorescence readings. In addition to SPCs (which may or may not contain cells), the systems herein are also suitable for other relatively big, suspended bioactive or living particles like organoids, spheroids, embryos, large single-cell organisms, cell colonies, oocytes, incompletely disaggregated tissue or cell clusters, and plant cells, which tend to be large and often difficult to disaggregate in light of their connective cell walls.

Creating a population of these large particles is very difficult using available methods. even if the selection criteria are basic (e.g. mean fluorescence intensity).

Furthermore, independent of size, imaging of flowing or in particular settled particles allows more subtle or sophisticated discrimination among target particle pools to identify particles for selection. In particular, by working with images of settled particles, one may identify features relevant for particle retention by looking at a sample or a subset of a sample as a whole rather than by making ongoing particle-by-particle assessments of a linear single file flow of particles. Thus, one may tailor selection parameters to select particles of a particular morphology or other particular parameters, but may also generate a population of selected particles as a proportion of the starting target particle pool, or in light of observations of this target pool. Thus, one may ensure, for example, that a target number of particles are selected from a target population by modulating stringency parameters, rather than screening through a target population only to find at the end of a run that parameters were too stringent and an insufficient number of particles were recovered.

Selected populations arising from the systems or methods herein may exhibit one or more of the following characteristics. Selected populations are in some cases selected to represent at least a set proportion of a target population, such as at least or at most 1%, 2%, 3%, 4%, 5%, 10%, 15%, 20%, 25%, 30%, 35%, 40%, 50% or more of an initial target population. This proportion is in some cases selected in combination with modulation of selection parameters such that the selected population nonetheless exhibits a uniform minimum particle trait.

Selected populations in some case exhibit a uniformity or uniform minimum value for one or more selection parameters. That is, some populations exhibit a uniform minimum parameter such that at least 60%, 70%, 80%, 90%, 95%, 96%, 97%, 98%, or 99% or more of the selected particles of the population exhibit the trait or combination of traits or ‘gate’, such as a minimum size, size within a range, density, minimum fluorescence, fluorescence withing a range, shape, number of cells within particle, morphology or types of cells withing a particle, or other feature.

As to uniformity, some populations exhibit a variation between lowest and highest value for a parameter or for each of more than one parameter of no more than 50%, 40%, 30%, 20%, 15%, 10%, 5%, or less than 5%, reflective of the precision with which selected populations may be isolated from target pools.

Selected populations in some cases comprise particles of a size or shape that is not conducive to traditional fluidics directed sorting such as microfluidics or FACs sorting. Selected particles in some cases have a minimum diameter of at least 10 μm, 20 μm, 30 μm, 40 μm, 50μm, 60 μm, 70 μm, 80 μm, 90 μm, 100 μm, 110 μm, 120 μm, 130 μm, 140 μm, 150 μm, or greater.

Selected populations benefit in some cases from the gentle biological conditions of particle capture and of particle release, such that at least 60%, 70%, 80%, 90%, 95%, 96%, 97%, 98%, or 99% or more of the selected particles of the population exhibit viability or biological activity, for example as a function of the total number of particles in the selected population, or as a function of the number of particles in the target pool exhibiting the viability or biological activity.

Selected populations are often generated without individual particles being subjected to particle specific microfluidic manipulation. Rather, target populations are often allowed to settle on a flow cell surface, in aggregate, imaged and then selected particles from among the target population and subjected to localized hydrogel formation so that they may be retained while remaining target pool or population constituents are discarded. Upon hydrogel degradation, the selected particles are released and collected in aggregate, such that selection does not rely upon individualized particle microfluidic manipulation.

Turning to the figures, one sees the following.

At FIG. 1A one sees a schematic of iterative particle capture consistent with the disclosure herein. A target pool is loaded into a chip, at left, and then subjected to iterative cycles of injection into the flow cell, imaging and targeted polymerization to capture identified targets from among the target pool image. These iterative cycles may be performed by flowing the target pool through a loop channel or course so that it returns to the input point, or by reversing flow such that the screened target pool flows back through the flow cell area to an initial point.

These iterative cycles may be performed successively on a closed system, or may be interrupted by any of a broad range of intervening treatment steps, from reintroducing a stain or introducing a second stain, to incubation of target pool microcapsules in culture medium to encourage proliferation of encapsulated single cells or small cell populations.

Subsequent to target capture, targets are released from the flow cell surface through degradation of the hydrogel, such as via enzymatic, biocompatible or even more harsh treatment such as head or acid or base treatment.

At FIG. 1B, one sees an alternative schematic of particle capture consistent with the disclosure herein. A target pool is loaded into a chip, at left, and then injected, in some cases completely, into a flow cell. The target pool is allowed to settle in the flow cell, and a camera images a portion of the flow cell. Identified particles are captured, such as by formation of a hydrogel at each particle so as to fix them in place. The camera or other imaging apparatus them moves to a new portion of the flow cell and the process is repeated until a portion or all of the target pool or flow cell has been imaged and selected members of the target pool have been fixed in place such as through hydrogel formation at the targets.

Subsequent to target capture, targets are released from the flow cell surface through degradation of the hydrogel, such as via enzymatic, biocompatible or even more harsh treatment such as bead or acid or base treatment.

At FIG. 2, one sees a cartoon of three steps in particle capture on a flow cell. The target pool is introduced at left onto a flow cell having a wider cross-sectional area than the introducing channel. The target pool is imaged, at center, and targets (red/dark) are identified for polymerization. These targets are subjected to local polymerization, fixing them to the flow cell, at right. At far right is the outflow channel, where unselected target pool constituents may be collected to be discarded as in FIG. 1B or for iterative re-screening as described in FIG. 1A, and where retained target contents (green/dark) are eventually recovered after release from the flow cell.

At FIG. 3, one sees examples of formed hydrogel spots (identified as labeled florescent) under various experimental conditions. These conditions include, at upper left, DexMAB, 0.1% LAP, 50 ms exposure, 4 mW; at upper right, DexMAB, 1% LAP, 50 ms exposure, 4 mW; at lower left, GelMA, 0.19% LAP, 50 ms exposure, 4 mW; and at lower right, GelMA, 0.1% LAP, 50 ms exposure, 4 mW. In all images, the large round areas mark plastic flow cell support features, while all images include a 250 μm scale bar. These images demonstrate successful hydrogel polymerization for a broad range of carrier compositions and concentrations.

At FIG. 4, one sees a schematic of a target pool flowing through a target capture arca. At top, one sees a cartoon of the LED illumination apparatus, comprising 740 nm illumination at 1.0W/cm2 in a 1×1 cm2 area. At bottom, one sees an exemplary flow cell channel cross section having pneumatics of 2 pumps and chip well seals. One sees that a target population is flowing from left to right across a flow cell, being illuminated and imaged as it passes. On further sees that the unselected target pool is captured in a reservoir at right. After completion of a screening round the remaining target pool may be discarded, flowed through a second flow cell, processed or subjected to off-device treatment such as culturing conditions, or in some cases flowed back leftward across the imaging and capture area to effect an additional round of selection. Optionally, a treatment may occur prior to the additional selection round, such as a wash or addition of a stain. Alternatively, the target pool may be collected and subjected to a treatment off the device, such as contacting to a culture medium. Following selection, target particles or target microcapsule contents may be released into either capture area, or drawn off the flow cell through a pipette access point not shown.

At FIG. 5A, one sees a target pool dataset onto which a retention parameter ‘gate’ is superimposed to facilitate identification of targets to be retained. The dataset is generated from quantified images of target pool members that have settled onto a flow cell surface. Imaged and quantified. The x-axis indicates particle diameter in pixels, ranging from 0 to 500 in intervals of 100. The y-axis indicates mean particle brightfield ‘brightness’, ranging from 0 to 250 in intervals of 50. The ‘gate’ is represented by the shaded box spanning below 100 pixels to slightly above 300 pixels, and a brightness of about 10 to almost 250. Particles withing the gate are designated for retention, while particles falling outside of the gate are discarded in this example. Gates may be variously predetermined or may be established de novo for a given image or set of images.

At FIG. 5B, one sees a schematic of an electronic control system architecture. The exemplary system presented is centralized around a motherboard and a Jetson Orin AGX, and further comprises cameras, a filter wheel, objective turret, Decor neopixels, HMI display, TP 15.4″, 1280×800, an interface for WiFi, ethernet, SD card or USB, 12V and 12V +/− power supplies, Control module comprising excitation LED controls, Laser scanner controls and illumination LED, and a pneumatics module of 2-4 piezo pumps. Alternate control system architectures are also consistent with the disclosure herein.

At FIG. 6 one sees a linear flow cell configuration, while FIGS. 10 and 11 depict a flow cell channel having branched ends. The systems present more flow cell area than may be imaged using the field of view of some imaging technologies. Furthermore, some image areas may become saturated with targets hydrogelled thereto. To address this, larger flow cell systems are provided, wherein the flow cell position may be adjusted such that different areas of the flow cell are presented to the imaging and laser or other energetics provision system. Not shown is a capture system or a loop channel to allow iterative rescreening of target pool populations.

At FIG. 7 one sees a cassette consistent with the disclosure herein. At left is a reservoir for a spacing or carrier solution, followed by a reservoir for depositing the sample target pool. At right one sees a pipette port for introduction of digestive enzymes and recovering captured particles or microcapsule contents, or target pool constituents to be re-assayed, as well as a waste reservoir.

At FIG. 8A, one sees monomer units of two exemplary polymers consistent with the disclosure herein. At left is a hyaluronic acid methacrylate monomer unit and at right is a gelatin methacrylate monomer unit. Polymers of each of these monomer units share a number of characteristics. In particular, they are semi-porous hydrogels. They may be induced to polymerization by exposure to oxidative stress, and they may be degraded under physiological conditions, such as through enzymatic or other gentle degradation.

At FIG. 8B, one sees an example of polymerized HAMA in a flow cell chamber. The scale bar represents 250 μm. The polymerized HAMA is configured in a 6×5 regular grid. Remnants of unpolymerized hydrogel appear as trails flowing away from the hydrogel array toward the bottom of the figure.

The image illustrates the level of precision attained by the mirror-directed laser polymerization of particle carrier compositions that facilitate retention of particles and generation of selected particle populations of the level of quality disclosed herein.

At FIG. 9, one sees an example of selectively captured microcapsules. At left, a population of microcapsules is flowed into a flow cell in a carrier comprising myaluronic acid methacrylate monomers and LAP, and imaged to assay for marker associated fluorescence, indicative of contents of interest. Exemplary markers include a nucleic amplification product or a bound antibody, such as a fluorophore labeled antibody.

Alternately, microcapsules may be imaged to assess their contents.

Target microcapsules are identified and subjected to local bursts of 405 nm light energy to trigger LAP induced oxidative stress in the microcapsule vicinities. The oxidative stress catalyzes local hyaluronic acid methacrylate polymerization that fixes the microcapsule in the immediate vicinity to the flow cell surface, while leaving the remaining microcapsules unaffected.

The flow cell is washed to remove unbound microcapsules, and the retained microcapsules are seen at right.

Unbound microparticles may be discarded or subjected to further treatment, such as incubation under growth conditions, and subjected to further analysis.

Bound particles may be subjected to one or more treatments, such as one or more of cell culturing, cell lysis, DNase treatment, reverse transcription, and library generation, for example, by successively washing the hydrogels in reagents to effect each step. The hydrogels are then degraded under biologically ‘gentle’ conditions and the microcapsule contents are released for bulk collection. In some cases, the microcapsules are individually barcoded, either by microcapsule barcoded beads or by contacting to position indicative oligos on the surface, such that even after bulking, they retain barcode information sufficient to map individual reads to a common microcapsule of origin.

At FIG. 10, one sees an exemplary microcapsule chip design. The chip is constructed from three layers: a plastic layer ‘A’ containing wells, a plastic layer ‘B’ containing microfluidic channels, and a flat and clear glass layer ‘C’, as shown in panel 1 at left.

Panel 2, center, shows the plastic layer ‘A’ in which an insertable pipette tip port is interfacing with a pipette tip. The plastic layer, and variants consistent with the disclosure herein, allows introduction of microcapsule or microparticle populations into the flow cell through, for example micropipette injection, as well as injection of reagents to be applied to hydrogel-captured microcapsules for manipulation of their contents and injection of an enzyme to catalyze hydrogel degradation under biologically ‘gentle’ conditions to release contents.

The four access wells of this layer correspond to the recovery port, upper left, waste well, lower left, buffer port, upper right, and sample inlet, lower right, presented in FIG. 11, below.

Panel 3, right, shows the plastic layer ‘B’ comprising a 10 pass zig-zag flow cell area available for particle capture. The image at FIG. 9. showing a funnel shaped upper bound of the capture area, corresponds to an upper end of one of the flow cell passes. Exemplary dimensions of the plastic layer ‘B’ are 50 mm across and 75 mm tall. The layer and variants thereof consistent with the disclosure herein present flow cell area that may be transparent to facilitate microparticle imaging or fluorescence detection, and to permit laser or light-induced catalysis of oxidative stress generation local to target microcapsules. In some cases, the available area comprises a lawn of oligos that vary positionally, such that nucleic acids tagged by local oligos and later sequenced may be mapped back to the position on the lawn corresponding to their oligo tagged sequences.

Notably, alternatives to plastic, alternative dimensions, and variants of the specific features of each layer are consistent with the disclosure herein.

At FIG. 11 one sees a more detailed view of the plastic layer ‘B’ of FIG. 10, above, illustrating selective channel blocking. Below the flow cell area indicated previously, the layer comprises a waste well, lower left, recovery port, upper left, sample inlet, lower right, and buffer inlet, upper right. These ports correspond to wells in the plastic well layer ‘A’ of FIG. 10. Sample is introduced through the sample inlet, and its passage through the flow cell passes, as well as washing and reagent delivery, is driven by carrier and reagents added through the buffer port. Flow through is directed to a waster well, while released microcapsule contents or recaptured microcapsules are collected at a recovery port.

After particles or microcapsules are captured on the chip, waste outlet and sample inlet channels can be blocked to prevent their contents contaminating the sample during captured particle recovery or other steps. The blocking is triggered by polymerizing the hydrogel withing channels. As the polymerization is enzyme-sensitive, the blocking can be reversed to reopen these channels as needed.

At left, the channels are unblocked, but sites where 405 nm LAP oxidative stress inducing illumination may be directed to block various channels is indicated. At right, the impact of these hydrogel blockages is indicated. Flow toward the sample inlet is blocked, while flow from the buffer inlet into the chip continues, both of which act to prevent sample inlet contamination with captured or processed product. Similarly, flow toward the waste well is blocked, such that departing fluid is directed to the recovery port.

This Fig. illustrates the manipulation of carrier flow by selective hydrogel formation.

At FIG. 12, one sees images and a schematic of hydrogel spot formation. Hydrogel spots form in response to laser-triggered oxidative stress evolution by LAP in a carrier if a polymerizable monomer. The hydrogel spot comprises a bleached central zone surrounded by a polymerized hydrogel zone. An image of a hydrogel spot indicating a pleached zone is seen at left, with a 10 um indicated for scale. At center, one sees a schematic of a hydrogel spot. The laser width is indicated at the middle of the spot, surrounded by a slightly larger diameter bleached zone, and a substantially larger polymerization hydrogel spot zone. At right one sees a second view of the hydrogel spot at right, this time indicating the polymerized hydrogel rather than the bleached zone, which is still visible at center.

Measured polymerized hydrogel diameters vary from about 25-38 um, while measured bleached zone diameter is about 15 um.

At FIG. 13, one sees data relating measured hydrogel spot diameter, indicated on the y-axis in um ranging from 25-45 in intervals of 5, as a function of LAP concentration indicated on the x-axis, at values of, from left to right, 0.1%, 0.25%, 0.5%, 1.0% and 1.5%, in a 2.5% hyaluronate methacrylate carrier, subjected to 20 ms light exposures.

One sees that, for a given concentration of hydrogel precursor in a carrier liquid, hydrogel diameter increases with LAP concentration. This allows one to use LAP concentration to calibrate hydrogel diameter, for example to accommodate target pool microparticle size.

### DEFINITIONS

As used herein, the term “about” in the context of a number refers to a range spanning 10% below the number to 10% above the number, while in the context of a range, the term refers to an extended range spanning from 10% below the listed lower limit to 10% above the upper listed limit.

As used herein, the phrase “at least one of” in the context of a group A, B, and C, for example, refers to sets including A, alone or with unlisted factors, B, alone or with unlisted factors, C, alone or with unlisted factors, A and B, alone or with unlisted factors, A and C, alone or with unlisted factors, B and C, alone or with unlisted factors, or A, B, and C, alone or with unlisted factors.

As used herein, the phrases “at least” or “no greater than” in the context of a following list of numbers are understood to apply distributively throughout the list, rather than only to the initial value of the list.

### EXAMPLES

Example 1. Hydrogel spot formation. Hydrogel spots (identified as labeled florescent) were formed under various experimental conditions. These conditions included, at upper left, DexMAB, 0.1% LAP, 50 ms exposure, 4 mW; at upper right, DexMAB, 1% LAP, 50 ms exposure, 4 mW; at lower left, GelMA, 0.1% LAP, 50 ms exposure, 4 mW; and at lower right, GelMA, 0.1% LAP, 50 ms exposure, 4 mW. In all images, the large round areas mark plastic flow cell support features, while all images include a 250 μm scale bar. These results, presented at FIG. 3, demonstrate successful hydrogel polymerization for a broad range of carrier compositions and concentrations.

Example 2. Particle capture for target enrichment. A population of microcapsules was flowed into a flow cell in a hyaluronic acid methacrylate/LAP carrier and assayed for fluorescence. Positive microcapsules were targeted with laser excitation emission to trigger localized hydrogel formation, fixing the positive microcapsules in place. Unbound microcapsules were washed out, leaving an enriched positive microcapsule population in place. See FIG. 9.

Example 3. Particle content release and collection. The bound microcapsules such as those of Example 2 can then be enzymatically treated to perform one or more manipulations, such as cell incubation, cell lysis, nucleic acid processing, library formation, and tag addition. Alternatively, tagging is in some cases effected subsequent to release, by contacting nucleic acid contents to oligos arrayed with positional information on the flow cell surface.

Microcapsules are released from the surface by enzymatic degradation of the external hydrogel and of the microcapsule exterior, releasing target microcapsule contents. Contents are then collected for pooled downstream analysis.

Example 4. Hydrogel spot size modulation.

Parameters were explored to assess the level of control that changes in various parameters have on hydrogel spot size. Results are presented at FIG. 13. A 2.5% hyaluronate methacrylate carrier, subjected to 20 ms light exposures, was assessed for hydrogel spot formation and size as a function of LAP concentration at values of 0.1%, 0.25%, 0.5%, 1.0% and 1.5%. Hydrogel microcapsules were repeatably formed under the full range of parameters, and that size increased with LAP concentration increase. The results indicate that hydrogel spot size may be modulated through adjustment of parameters herein, such that spot size mat be calibrated to particle or microcapsule size. Notably, in some cases a hydrogel spot size entirely encompasses the particle which it selects or tags to a surface, while in other cases the hydrogel spot covers over at least part of the at least one imaged target particle (such as at least, at most, or about 20%, 30%, 40%, 50%, 75%, 90%, 95%, 99% pr 100%, or a number spanned by or outside of this range) so as to fix, or thereby fixing the at least one imaged target particle at a position on the flow cell.

We claim:

1. A method of sorting particles, the method comprising: introducing a population of particles into a flow cell; identifying at least one target particle among at least some particles of the population of particles; selectively polymerizing a hydrogel over at least part of the at least one target particle so as to fix the at least one target particle at a position on the flow cell.

2. The method of claim 1, wherein identifying at least one target particle comprises capturing images of at least some particles of the population, identifying a target particle image among the images of the at least some particles, and identifying a particle corresponding to the target particle image as a target particle.

3-4. (canceled)

5. The method of claim 2, wherein the target particle image exhibits a target particle morphology.

6. (canceled)

7. The method of claim 1, wherein identifying at least one target particle comprises assaying the at least some of the particles for a signal output indicative of a target particle, and identifying a particle corresponding to the signal output.

8-15. (canceled)

16. The method of claim 1, comprising collecting constituents of the population of particles not fixed to a surface of the flow cell.

17. The method of claim 16, comprising re-introducing the constituents of the population of particles not fixed to the surface into the flow cell.

18. The method of claim 17 wherein, prior to reintroducing, the population of particles not fixed to the surface is subjected to a condition.

19-22. (canceled)

23. The method of claim 1, wherein the population of particles comprises cells.

24-25. (canceled)

26. The method of claim 1, wherein the population of particles comprises microcapsules.

27. The method of claim 26, wherein at least some of the microcapsules contain nucleic acids.

28. The method of claim 26, wherein at least some of the microcapsules contain living cells.

29. The method of claim 28, wherein the living cells are present at at least one cell per microcapsule in at least some of the microcapsules.

30. The method of claim 28, wherein the living cells are capable of dividing.

31. The method of claim 28, wherein the living cells are obtained from a heterogeneous cell population.

32. The method of claim 31, wherein the heterogeneous cell population is obtained at least in part from a tumor.

33. The method of claim 31, wherein the heterogeneous cell population is obtained at least in part from a circulating fluid.

34. The method of claim 31, wherein the heterogeneous cell population is obtained at least in part from blood.

35-46. (canceled)

47. The method of claim 1, wherein identifying at least one imaged target particle comprises comparing the image of the imaged target particle to a database.

48. The method of claim 1, wherein identifying at least one imaged target particle comprises comparing the image of the imaged target particle to another image of the population of particles.

49. The method of claim 1, wherein selectively polymerizing a hydrogel over at least part of the at least one imaged target particle comprises providing a laser pulse to polymerize over the imaged target particle.

50-117. (canceled)
